(19) United States
(12) Patent Application Publication
(10) Pub. No.: US 2013/0178534 A1
(43) Pub. Date: Jul. 11, 2013

(54) N-Nonanoylvanillylamine as an agent for reducing the appetite, as an agent for imparting a feeling of fullness and as a mood enhancer, and corresponding substance mixtures, orally consumable products and methods

(71) Applicant: Symrise AG, Holzminden (DE)

(72) Inventors: Jakob Ley, Holzminden (DE); Gerhard Krammer, Holzminden (DE); Veronika Somoza, Weidling (AT); Barbara Rohm, Wien (AT); Mark Somoza, Weidling (AT)

(73) Assignee: Symrise AG, Holzminden (DE)

(21) Appl. No.: 13/736,414

(22) Filed: Jan. 8, 2013

Related U.S. Application Data
(60) Provisional application No. 61/584,974, filed on Jan. 10, 2012.

(30) Foreign Application Priority Data
Jan. 10, 2012 (EP) 12150550.7-2114

(51) Int. Cl. A61K 31/165; A61K 45/06; C07C 233/22

(52) U.S. Cl. CPC A61K 31/165; C07C 233/22; A61K 45/06
USPC 514/625; 554/111

(57) ABSTRACT

There is described primarily N-nonanoylvanillylamine for use in a therapeutic method as (a) an agent for reducing the appetite and/or (b) an agent for imparting a feeling of fullness and/or (c) a mood enhancer, as well as the non-therapeutic use of N-nonanoylvanillylamine as (a) an agent for reducing the appetite and/or (b) an agent for imparting a feeling of fullness and/or (c) a mood enhancer. The non-therapeutic use of corresponding substance mixtures is also further described. Finally, the invention relates also to orally consumable products (in particular foodstuffs, feeds and medicaments) comprising N-nonanoylvanillylamine, wherein the N-nonanoylvanillylamine is present in a concentration that (a) reduces the appetite and/or (b) brings about a feeling of fullness and/or (c) enhances the mood, but which concentration is low.

The invention relates primarily to N-nonanoylvanillylamine for use in a therapeutic method as (a) an agent for reducing the appetite and/or (b) an agent for imparting a feeling of fullness and/or (c) a mood enhancer, and to the non-therapeutic use of N-nonanoylvanillylamine as (a) an agent for reducing the appetite and/or (b) an agent for imparting a feeling of fullness and/or (c) a mood enhancer.

The invention relates further to a method of (a) reducing the appetite and/or (b) imparting a feeling of fullness and/or (c) enhancing the mood. The invention further includes substance mixtures for use in specific therapeutic methods, comprising N-nonanoylvanillylamine and one or more further substances, as (a) agents for reducing the appetite and/or (b) agents for imparting a feeling of fullness and/or (c) mood enhancers. The invention relates also to the non-therapeutic use of corresponding substance mixtures. Finally, the invention relates also to orally consumable products (in particular foodstuffs, feeds and medicaments) comprising N-nonanoylvanillylamine, wherein the N-nonanoylvanillylamine is present in a concentration that (a) reduces the appetite and/or (b) brings about a feeling of fullness and/or (c) enhances the mood, but which concentration is low.

The frequent occurrence of permanent excess weight caused by a lack of exercise and/or excessive food intake can lead to chronic disorders such as obesity, insulin resistance, impaired lipid metabolism and/or hypertension, the serious secondary diseases thereof type II diabetes, arteriosclerosis, heart attack or stroke, and accordingly ultimately to early death. A high content of, in particular, readily metabolisable carbohydrates, proteins and especially fats in food leads to the formation of fat deposits and can ultimately contribute considerably to the above-mentioned problems. In order to limit the intake of such food constituents, especially fats and sweet carbohydrates (sugars), for which there is often a hedonic preference, their content in reduced-calorie foodstuffs, so-called “light products”, is often greatly reduced and replaced by substitute substances (thickeners for fats, non-caloric sweeteners instead of sugars).

When “light products” are consumed, it can often happen that a product which, owing to a clever formulation, has a hedonic value comparable to that of the energy-rich original product, is consumed in greater amounts and, in the worst case, the intake of calorically relevant food constituents is then even increased, and the aim of reducing the amount of calories ingested is accordingly not achieved.

In order to counteract increased ingestion of calorically relevant food constituents, it has for a long time been desired to find food constituents, in particular flavourings, that are rated safe, are already allowed and are generally accepted, which are able to reduce the feeling of hunger and the natural appetite and/or correspondingly to increase the feeling of fullness. It is known that it is possible to influence the appetite negatively and the fullness positively by increasing the release of dopamine and serotonin in certain areas of the brain, while at the same time providing exposure to nutrients from the orally consumable products (in particular foodstuffs) that are ingested, and by inducing leptin receptor and serotonin receptor proteins.

In addition to effects on the appetite and fullness, the mood of the consumer can also be influenced positively. For example, foodstuffs are already known that comprise dopamine and serotonin and have a mood-enhancing action. If serotonin is present in sufficient amounts in the brain, it imparts a positive mood, good concentration capacity and optimism. Low serotonin levels, on the other hand, can lead to irritability, disturbed sleep, an inability to concentrate and depression. Dopamine affects alertness, pleasure and mental clarity. A dopamine deficiency manifests itself inter alfa in apathy, an inability to love and a lack of remorse.

Ingestion of serotonin and dopamine via food increases the concentrations of serotonin and dopamine in the blood and is probably also available for interactions with receptors in the brain. In order to increase the serotonin and dopamine concentrations in the brain, it is advantageous to stimulate the release of serotonin and dopamine in the brain.

Studies have shown that pure capsaicin, the most important pungent substance in the chilli pepper (Capsicum anuum), can exhibit an appetite-reducing and fullness-increasing effect (Smeets, A. J. P. G.; Westerterp-Plantenga, M., Capsaicin. In Weight Control and Slimming Ingredients in Food Technologies, Cho, Susan S. (Ed.), pp. 201-211, Wiley-Blackwell; Ames, Iowa, 2010). This observed effect is presumably based on the fact that capsaicin imparts a sensation of heat when ingested orally.

However, the use of capsaicin in foodstuffs is not permitted in the European Union (it was deleted from the Community Flavoring List in 2004), because the compound was negatively rated as having genotoxic potential (European Food Safety Authority (EFSA), P., Italy, Opinion of the Scientific Committee on Food on Capsaicin. European Commission 2002, (SDF/CS/FLAV/FLAVOUR/8 ADD1 Final)). In addition, capsaicin is often very difficult to use in foodstuffs because it has a low taste threshold and high potency as a pungent substance (16,000,000 Scoville units, see http://en.wikipedia.org/wiki/Capsaicin; version of the entry last amended on 11Nov. 2011, 21:02). In addition, owing to the high price of the pure substance, capsaicin is used almost exclusively in the form of a capsicum extract which, in addition to further pungent substances, also comprises residues of other flavourings that taste or smell of capsicum and therefore has only limited suitability for widespread use.

The primary object of the present invention was to find a flavouring (preferably permitted under foodstuffs law) which—being similar to capsaicin in terms of its action—can be used (a) as an agent for reducing the appetite and/or (b) as an agent for imparting a feeling of fullness and/or (c) as a mood enhancer.

The object is achieved by N-none noylvanillylamine for use in a therapeutic method

(a) as an agent for reducing the appetite,
preferably for reducing the caloric intake and hence preferably for therapeutic weight reduction,

and/or

(b) as an agent for imparting a feeling of fullness,
preferably for reducing the caloric intake and hence preferably for therapeutic weight reduction,

and/or

(c) as a mood enhancer.

The present invention relates likewise to the non-therapeutic use of N-nonanoylvanillylamine

(a) as an agent for reducing the appetite,
preferably for reducing the caloric intake and hence preferably for non-therapeutic weight reduction,

and/or

(b) as an agent for imparting a feeling of fullness,
preferably for reducing the caloric intake and hence preferably for non-therapeutic weight reduction,

and/or

(c) as a mood enhancer.

The present invention relates likewise to the cosmetic use of N-nonanoylvanillylamine as an agent for reducing the appetite and/or an agent for imparting a feeling of fullness, preferably in each case for reducing the caloric intake and hence preferably for cosmetic weight reduction.

N-Nonanoylvanillylamine is a known and permitted flavouring (Flavis number 16.006; EFSA: has been rated in FGE.86 as having “no safety concern”; CAS number 2444-46-4) and is already in use in a large number of foodstuffs for achieving pronounced pungency. The stimulant threshold of N-nonanoylvanillylamine is 0.054 mg/kg, and the detection threshold is 0.107 mg/kg (see Kollmannsberger, H. Inhaltsstoffzusammensetzung and sensorische QuaRat von 20 Kultivaren verschiedener Capsicum-Arten. Dissertation, Technical University of Munich, Weihenstephan, 2007, page 9). Typical use concentrations in foodstuffs are from 1 to 10 mg/kg and for chewing gum 50 mg/kg (see Flavor Base 2010, entry “Nonanoyl hydroxymethylbenzylamide”, Record #2174, Leffingwell & Associates).

N-Nonanoylvanillylamine is also known under the names pseudocapsaicin, N-((4-hydroxy-3-methoxyphenyl)methyl)nonanamide, pelargonic acid vanillylamide and nonivamide. N-Nonanoylvanillylamine (compound I) is shown in the following diagram for clarification:

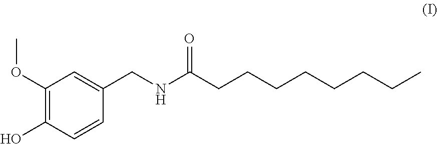 [SMILES: CCCCCCCCC(=O)NCc1ccc(O)c(OC)c1]

As already noted above in relation to the example of capsaicin, the use of pungent-tasting compounds is not always possible without problems. Pungency is often not desirable in orally consumable products (in particular foodstuffs, feeds and medicaments), and orally consumable products with too pungent a taste are frequently rejected by the consumer. An additional object of the present invention was, therefore, to find a substance which has the properties mentioned in the statement of the object and can be used in such a manner that it achieves the object according to the invention and at the same time does not or does not significantly influence the organoleptic properties of an orally consumable product (in particular foodstuff, feed or medicament) and accordingly can be used in an orally consumable product without having a sensory or sensorially significant influence thereon.

Surprisingly, it has been shown that N-nonanoylvanillylamine is capable
(a) in a concentration range of only from 0.01 μM to 10 μM (from 0.003 mg/kg to 3 mg/kg), of stimulating the release of serotonin in neurons by a maximum of up to about 270%, compared with the respective control,

and
(b) in a concentration of only approximately 0.03 mg/kg (which corresponds to 0.1 μM), of stimulating the release of dopamine following stimulation with acetylcholine by up to about 700%, compared with the respective control,
   that is to say in each case in a concentration that is both significantly below the threshold values and also far below typical use concentrations in orally consumable products (in particular foodstuffs, feeds or medicaments). See in this connection the examples hereinbelow.

The lipophilicity of N-nonanoylvanillylamine generally effects good bioavailability, that is to say good resorption from the gastrointestinal tract into the bloodstream. Accordingly, the above-mentioned biological effects occur in vivo in particular even at use concentrations of N-nonanoylvanillylamine that are below the taste detection threshold, that is to say below 0.1 mg/kg, and on ingestion of conventional amounts of orally consumable products (in particular of foodstuffs, feeds and medicaments).

Preferably, N-nonanoylvanillylamine for use according to the invention is used in a therapeutic method or in a non-therapeutic or cosmetic use according to the invention, wherein the N-nonanoylvanillylamine is used as a constituent of an orally consumable product (in particular of a foodstuff, feed or medicament), wherein
the N-nonanoylvanillylamine is present in a concentration of 1 mg/kg or less, based on the total mass of the orally consumable product, preferably in a concentration of 0.50 mg/kg or less, particularly preferably in a concentration of 0.10 mg/kg or less, most particularly preferably in a concentration of 0.05 mg/kg or less, and/or
the orally consumable product comprises no capsaicin, preferably no capsaicin and, apart from N-nonanoylvanillylamine, no further capsaicinoids.

Preference is given to an orally consumable product according to the invention selected from the group comprising foodstuffs (in particular liquid or solid foodstuffs, including semi-finished products), feeds and medicaments (pharmaceutical preparations).

Within the context of the present text, the term “foodstuffs” covers a plurality of products. The term foodstuffs includes in particular products as are discussed hereinbelow in connection with foodstuffs according to the invention.

The term “foodstuffs” covers in particular products that are foodstuffs according to REGULATION (EC) No. 178/2002 OF THE EUROPEAN PARLIAMENT AND OF THE COUNCIL of 28 Jan. 2002. According to this regulation, “foodstuffs” are any substances or products which in the processed, partially processed or unprocessed state are intended to be, or reasonably expected to be, ingested by humans. “Foodstuffs” also include drinks, chewing gum and any substance—including water—intentionally added to the foodstuff during its manufacture, preparation or treatment.

Within the context of the present text, the term “feed” covers all forms of animal food. A large number of the foodstuffs mentioned hereinbelow can also be used as feeds.

Within the context of the present text, the term “medicament” covers substances or substance compositions which are intended as agents having properties for curing or for preventing human or animal diseases or which can be used in or on the human or animal body or administered to a human or animal in order to restore, correct or influence either human or animal physiological functions by a pharmacological, immunological or metabolic action, or to produce a medical diagnosis. Medicaments can be used in particular cases for non-therapeutic, in particular cosmetic, purposes.

It will be appreciated that foodstuffs or feeds can be converted into corresponding medicaments by the addition of substances or substance compositions which are intended as agents having properties for curing or for preventing human or animal diseases.

A further aspect of the present invention is the use of N-nonanoylvanillylamine in a therapeutic method

(a) as an agent for reducing the appetite,
preferably for reducing the caloric intake and hence preferably for therapeutic weight reduction,

and/or

(b) as an agent for imparting a feeling of fullness,
preferably for reducing the caloric intake and hence preferably for therapeutic weight reduction,

and/or

(c) as a mood enhancer.

The present invention relates also to a method

(a) for the non-therapeutic reduction of the appetite,
preferably for the non-therapeutic reduction of the caloric intake and hence preferably for non-therapeutic weight reduction,

and/or

(b) for the non-therapeutic imparting of a feeling of fullness,
preferably for the non-therapeutic reduction of the caloric intake and hence preferably for non-therapeutic weight reduction,

and/or

(c) for enhancing the mood,

comprising the following step:
administration of N-nonanoylvanillylamine to a human or animal subject in an amount that (i) reduces the appetite and/or (ii) brings about a feeling of fullness and/or (iii) enhances the mood.

The present invention relates likewise to a substance mixture comprising N-nonanoylvanillylamine and one or more further substances, for use in a therapeutic method

(a) as an agent for reducing the appetite,
preferably for reducing the caloric intake and hence preferably for therapeutic weight reduction,

and/or

(b) as an agent for imparting a feeling of fullness,
preferably for reducing the caloric intake and hence preferably for therapeutic weight reduction,

and/or

(c) as a mood enhancer,

wherein in the therapeutic method the N-nonanoylvanillylamine contained in the substance mixture is used in such a manner that it reduces the appetite and/or causes a feeling of fullness and/or enhances the mood.

The present invention relates likewise to a non-therapeutic use of a substance mixture comprising N-nonanoylvanillylamine and one or more further substances

(a) as an agent for reducing the appetite,
preferably for reducing the caloric intake and hence preferably for non-therapeutic weight reduction,

and/or

(b) as an agent for imparting a feeling of fullness,
preferably for reducing the caloric intake and hence preferably for non-therapeutic weight reduction,

and/or

(c) as a mood enhancer,

wherein the N-nonanoylvanillylamine contained in the substance mixture is used in such a manner that it reduces the appetite and/or causes a feeling of fullness and/or enhances the mood.

In a preferred substance mixture according to the invention for use and in a preferred use according to the invention of a substance mixture, the substance mixture is in each case an orally consumable product (in particular foodstuff, feed or medicament) or a constituent of an orally consumable product (in particular foodstuff, feed or medicament),

wherein the orally consumable product comprises the N-nonanoylvanillylamine preferably in a concentration of less than 1 mg/kg, based on the total mass of the orally consumable product,

preferably in a concentration of 0.50 mg/kg or less, particularly preferably in a concentration of 0.10 mg/kg or less, most particularly preferably in a concentration of 0.05 mg/kg or less.

In a particularly preferred substance mixture according to the invention for use and in non-therapeutic uses according to the invention of a substance mixture or in cosmetic uses according to the invention of a substance mixture, the substance mixture in each case comprises a maximum amount of capsaicin such that the ratio by mass capsaicin: N-nonanoylvanillylamine is 100:1, preferably 50:1, more preferably 20:1, more preferably 1:1, more preferably 1:10 and more preferably 1:100,

wherein the substance mixture preferably comprises no capsaicin,

more preferably no capsaicin and, apart from N-nonanoylvanillylamine, no further capsaicinoids.

A further aspect of the present invention relates to the use of a substance mixture comprising N-nonanoylvanillylamine and one or more further substances in a therapeutic method

(a) as an agent for reducing the appetite,
preferably for reducing the caloric intake and hence preferably for therapeutic weight reduction,

and/or

(b) as an agent for imparting a feeling of fullness,
preferably for reducing the caloric intake and hence preferably for therapeutic weight reduction,

and/or

(c) as a mood enhancer,

wherein in the therapeutic method the N-nonanoylvanillylamine contained in the substance mixture is used in such a manner that it (i) reduces the appetite and/or (ii) causes a feeling of fullness and/or (iii) enhances the mood.

In a particularly preferred substance mixture according to the invention for use and in particularly preferred non-therapeutic uses according to the invention of a substance mixture and in particularly preferred cosmetic uses according to the invention of a substance mixture, the further substances are preferably spray-dried substances which comprise constituents suitable for consumption, solid carriers and optionally specific flavourings or flavour compositions.

In further particularly preferred substance mixtures according to the invention for use and in particularly preferred uses according to the invention of a substance mixture, the further substances are solid carriers suitable for consumption.

Advantageous carriers in those preferred (preferably spray-dried) substance mixtures according to the invention are silicon dioxide (silica, silica gel), carbohydrates and/or carbohydrate polymers (polysaccharides), cyclodextrins, starches, degraded starches (starch hydrolysates), chemically or physically modified starches, modified celluloses, gum arabic, ghatti gum, tragacanth, karaya, carrageenan, guar gum, locust bean gum, alginates, pectin, inulin or xanthan gum. Preferred starch hydrolysates are maltodextrins and dextrins.

Preferred carriers are silicon dioxide, gum arabic and maltodextrins, preference in turn being given to maltodextrins having DE values in the range from 5 to 20. It is not important which plant originally supplied the starch for the preparation of the starch hydrolysates. Maize-based starches as well as starches from tapioca, rice, wheat or potatoes are suitable and readily available. The carriers can also serve as flow aids, such as, for example, silicon dioxide.

The substance mixtures according to the invention, which, in addition to the N-nonanoylvanillylamine to be used according to the invention, also comprise one or more solid carriers, can be produced, for example, by mechanical mixing procedures, wherein communication of the particles can also be carried out at the same time, or by means of spray drying. Preference is given to compositions according to the invention that comprise solid carriers and are produced by means of spray drying; with regard to spray drying, reference is made to U.S. Pat. No. 3,159,585, U.S. Pat. No. 3,971,852, U.S. Pat. No. 4,532,145 or U.S. Pat. No. 5,124,162.

Preferred substance mixtures according to the invention that comprise carriers and have been produced by spray drying have a mean particle size in the range from 30 to 300 μm and a residual moisture content of less than or equal to 5 wt. %.

A substance mixture according to the invention is preferably a (preferably spray-dried) substance mixture which, in addition to

(i) an effective amount of N-nonanoylvanillylamine and

(ii) solid carriers,

additionally comprises

(iii) one or more volatile flavourings,

or consists of the mentioned components. As regards preferred compounds and mixtures, the comments made above apply correspondingly. Preferably, the substance mixture comprises no capsaicin and, apart from N-nonanoylvanillylamine, no further capsaicinoids.

The or at least one of the volatile flavourings of component (iii) is preferably a sensorially active component having a vapour pressure of greater than or equal to 0.01 Pa at 25° C., preferably a vapour pressure of greater than or equal to 0.025 Pa at 25° C., and it is preferably used in a concentration that is greater than its stimulant threshold. A large number of volatile flavourings have a vapour pressure of greater than or equal to 1 Pa at 25° C.; these flavourings are regarded as preferred for use in substance mixtures according to the invention.

Examples of flavourings of component (iii) which can be a constituent of the substance mixture will be found, for example, in H. Surburg, J. Panten, Common Fragrance and Flavor Materials, 5th Ed., Wiley-VCH, Weinheim 2006.

Flavourings of component (iii) can be used in the form of flavour compositions, which in turn can be used in the form of reaction flavourings (Maillard products) and/or extracts or ethereal oils of plants or plant parts or fractions thereof, with the exclusion of capsicum species.

In a further aspect, the present invention relates to an orally consumable product (in particular foodstuff, feed and medicament) comprising N-nonanoylvanillylamine and one or more further substances, wherein the N-nonanoylvanillylamine is present
in a concentration that reduces the appetite and/or brings about a feeling of fullness and/or

in a concentration that enhances the mood

and at the same time
in a concentration of 1 mg/kg or less, based on the total mass of the orally consumable product, preferably in a concentration of 0.50 mg/kg or less, particularly preferably in a concentration of 0.10 mg/kg or less, most particularly preferably in a concentration of 0.05 mg/kg or less,

and at the same time
in a concentration of at least 0.001 mg/kg or more, based on the total mass of the orally consumable product, preferably in a concentration of 0.005 mg/kg or more, most particularly preferably in a concentration of 0.01 mg/kg or more, and

the orally consumable product comprises no capsaicin,

preferably comprises no capsaicin and, apart from N-nonanoylvanillylamine, no further capsaicinoids.

Particular preference is given to an orally consumable product according to the invention which is a foodstuff, feed and/or medicament.

In order to assist a reduction in the body weight of a consumer, it has been found to be expedient to limit the caloric intake. This can be achieved, on the one hand, in that the use according to the invention of N-nonanoylvanillylamine in orally consumable products (in particular foodstuffs, feeds and medicaments) imparts a feeling of fullness and at the same time reduces the appetite. As a result, the consumer is made to limit the consumption of energy-rich products. The caloric intake can additionally be reduced if orally consumable products according to the invention (in particular foodstuffs, feeds and medicaments) which in turn already have a low energy density are offered for consumption.

A preferred orally consumable product (in particular foodstuff, feed and medicament) according to the invention contains not more than 200 kcal/100 g of the orally consumable product, preferably not more than 100 kcal/100 g, particularly preferably not more than 40 kcal/100 g.

Preferred orally consumable products (in particular foodstuffs, feeds or medicaments) according to the invention are any preparations or compositions which are suitable for consumption and are used for nutrition, oral care or enjoyment purposes, and are generally products which are intended to be introduced into the human or animal oral cavity, to remain there for a certain time and then either be eaten (e.g. ready-to-eat foodstuffs or feeds, see also hereinbelow) or removed from the oral cavity again (e.g. chewing gums or oral care products or medicinal mouthwashes). Such products include any substances or products which in the processed, partially processed or unprocessed state are to be ingested by humans or animals. They also include substances which are added to orally consumable products (in particular foodstuffs, feeds and medicaments) during their manufacture, preparation or treatment and which are intended to be introduced into the human or animal oral cavity.

The orally consumable products (in particular foodstuffs, feeds and medicaments) according to the invention also include substances which in the unchanged, treated or prepared state are to be swallowed by a human or animal and then digested; in this respect, the orally consumable products according to the invention also include casings, coatings or other encapsulations which are to be swallowed at the same time or which may be expected to be swallowed. The expression “orally consumable product” covers ready-to-eat foodstuffs and feeds, that is to say foodstuffs or feeds that are already complete in terms of the substances that are important for the taste. The expressions “ready-to-eat foodstuff' and “ready-to-eat feed” also include drinks as well as solid or semi-solid ready-to-eat foodstuffs or feeds. Examples which may be mentioned are frozen products, which must be thawed and heated to eating temperature before they are eaten. Products such as yoghurt or ice-cream as well as chewing gums or hard caramels are also included among the ready-to-eat foodstuffs or feeds.

Preferred orally consumable products (in particular foodstuffs and feeds) according to the invention also include “semi-finished products”. Within the context of the present text, a semi-finished product is to be understood as being an orally consumable product which, because of a very high content of flavourings and taste-imparting substances, is unsuitable for use as a ready-to-eat orally consumable product (in particular foodstuff or feed). Only by mixing with at least one further constituent (e.g. by reducing the concentration of the flavourings and taste-imparting substances in question) and optionally further process steps (e.g. heating, freezing) is the semi-finished product converted into a ready-to-eat orally consumable product (in particular foodstuff or feed). Examples of semi-finished products which may be mentioned here are packet soups, extracts for baking and instant pudding powders.

Orally consumable products (in particular foodstuffs, feeds or medicaments) according to the invention also include “oral care products”. Within the scope of the invention, an oral care product (also called oral hygiene product or oral hygiene preparation) is understood as being a formulation known to the person skilled in the art for cleaning and caring for the oral cavity and the pharyngeal space and for freshening the breath. Care of the teeth and gums is expressly included here. Forms of administration of conventional oral hygiene formulations are in particular creams, gels, pastes, foams, emulsions, suspensions, aerosols, sprays, as well as capsules, granules, pastilles, tablets, sweets or chewing gums, whereby this list is not to be understood as being limiting for the purposes of this invention.

An orally consumable product (in particular foodstuff or feed) according to the invention preferably comprises one or more preparations for nutrition or enjoyment purposes. These include in particular (reduced-calorie) baked goods (e.g. bread, dry biscuits, cakes, other baked articles), confectionery (e.g. chocolates, chocolate bars, other products in bar form, fruit gums, dragées, hard and soft caramels, chewing gum), non-alcoholic drinks (e.g. cocoa, coffee, green tea, black tea, (green, black) tea drinks enriched with (green, black) tea extracts, rooibos tea, other herbal teas, fruit-containing soft drinks, isotonic drinks, refreshing drinks, nectars, fruit and vegetable juices, fruit or vegetable juice preparations), instant drinks (e.g. instant cocoa drinks, instant tea drinks, instant coffee drinks), meat products (e.g. ham, fresh sausage or raw sausage preparations, spiced or marinated fresh or salt meat products), eggs or egg products (dried egg, egg white, egg yolk), cereal products (e.g. breakfast cereals, muesli bars, precooked ready-to-eat rice products), dairy products (e.g. full-fat or reduced-fat or fat-free milk drinks, rice pudding, yoghurt, kefir, cream cheese, soft cheese, hard cheese, dried milk powder, whey, butter, buttermilk, partially or completely hydrolysed milk-protein-containing products), products made from soy protein or other soybean fractions (e.g. soy milk and products produced therefrom, drinks containing isolated or enzymatically treated soy protein, drinks containing soy flour, preparations containing soy lecithin, fermented products such as tofu or tempeh or products produced therefrom and mixtures with fruit preparations and optionally flavours), fruit preparations (e.g. jams, sorbets, fruit sauces, fruit fillings), vegetable preparations (e.g. ketchup, sauces, dried vegetables, frozen vegetables, precooked vegetables, boiled-down vegetables), snacks (e.g. baked or fried potato crisps or potato dough products, maize- or groundnut-based extrudates), fat- and oil-based products or emulsions thereof (e.g. mayonnaise, remoulade, dressings, in each case full-fat or reduced-fat), other ready-made dishes and soups (e.g. dried soups, instant soups, precooked soups), spices, spice mixtures and in particular seasonings which are used, for example, in the snacks field, sweetener preparations, tablets or sachets, other preparations for sweetening or whitening drinks or other foods. The preparations within the scope of the invention can also be used in the form of semi-finished products for the production of further preparations for nutrition or enjoyment purposes. The preparations within the scope of the invention can also be in the form of capsules, tablets (uncoated and coated tablets, e.g. enteric coatings), dragées, granules, pellets, solids mixtures, dispersions in liquid phases, in the form of emulsions, in the form of powders, in the form of solutions, in the form of pastes, or in the form of other preparations which can be swallowed or chewed, and in the form of food supplements.

Particular preference is given to reduced-calorie confectionery (e.g. muesli bar products, fruit gums, dragées, hard and soft caramels, chewing gum), non-alcoholic drinks (e.g. cocoa, green tea, black tea, (green, black) tea drinks enriched with (green, black) tea extracts, rooibos tea, other herbal teas, fruit-containing soft drinks, isotonic drinks, refreshing drinks, nectars, fruit and vegetable juices, fruit or vegetable juice preparations), instant drinks (e.g. instant cocoa drinks, instant tea drinks), cereal products (e.g. breakfast cereals, muesli bars, precooked ready-to-eat rice products), dairy products (e.g. full-fat or reduced-fat or fat-free milk drinks, rice pudding, yoghurt, kefir, dried milk powder, whey, buttermilk, partially or completely hydrolysed milk-protein-containing products), products made from soy protein or other soybean fractions (e.g. soy milk and products produced therefrom, drinks containing isolated or enzymatically treated soy protein, drinks containing soy flour, preparations containing soy lecithin, fermented products such as tofu or tempeh or products produced therefrom and mixtures with fruit preparations and optionally flavours), sweetener preparations, tablets or sachets, other preparations for sweetening or whitening drinks or other foods.

Very particular preference is given to reduced-calorie or calorie-free confectionery (e.g. muesli bar products, fruit gums, dragées, hard caramels, chewing gum), non-alcoholic drinks (e.g. green tea, black tea, (green, black) tea drinks enriched with (green, black) tea extracts, rooibos tea, other herbal teas, fruit-containing low-sugar or sugar-free soft drinks, isotonic drinks, nectars, fruit and vegetable juices, fruit or vegetable juice preparations), instant drinks (e.g. instant (green, black, rooibos, herbal) tea drinks), cereal products (e.g. low-sugar or sugar-free breakfast cereals, muesli bars), dairy products (e.g. reduced-fat or fat-free milk drinks, yoghurt, kefir, whey, buttermilk), products made from soy protein or other soybean fractions (e.g. soy milk and products produced therefrom, drinks containing isolated or enzymatically treated soy protein, drinks containing soy flour, preparations containing soy lecithin, or products produced therefrom and mixtures with fruit preparations and optionally flavours) or sweetener preparations, tablets or sachets.

The preparations can also be in the form of capsules, tablets (uncoated and coated tablets, e.g. enteric coatings), dragées, granules, pellets, solids mixtures, dispersions in liquid phases, in the form of emulsions, in the form of powders, in the form of solutions, in the form of pastes, or in the form of other preparations which can be swallowed or chewed, for example in the form of food supplements.

The semi-finished products are generally used for the production of ready-to-use or ready-to-eat preparations for nutrition or enjoyment purposes.

Further constituents of a ready-to-eat preparation or semi-finished product for nutrition or enjoyment purposes can be conventional base substances, auxiliary substances and additives for foods or enjoyment foods, for example water, mixtures of fresh or processed, vegetable or animal base or raw substances (e.g. raw, roast, dried, fermented, smoked and/or boiled meat, bone, cartilage, fish, vegetables, herbs, nuts, vegetable juices, vegetable pastes or mixtures thereof), digestible or non-digestible carbohydrates (e.g. sucrose, maltose, fructose, glucose, dextrins, amylose, amylopectin, inulin, xylans, cellulose, tagatose), sugar alcohols (e.g. sorbitol, erythritol), natural or hardened fats (e.g. tallow, lard, palm fat, cocoa fat, hardened vegetable fat), oils (e.g. sunflower oil, groundnut oil, maize germ oil, olive oil, fish oil, soya oil, sesame oil), fatty acids or their salts (e.g. potassium stearate), proteinogenic or non-proteinogenic amino acids and related compounds (e.g. γ-aminobutyric acid, taurine), peptides (e.g. glutathione), natural or processed proteins (e.g. gelatin), enzymes (e.g. peptidases), nucleic acids, nucleotides, taste correctors for unpleasant taste impressions, further taste modulators for further, generally not unpleasant taste impressions, other taste-modulating substances (e.g. inositol phosphate, nucleotides such as guanosine monophosphate, adenosine monophosphate or other substances such as sodium glutamate or 2-phenoxypropionic acid), emulsifiers (e.g. lecithins, diacylglycerols, gum arabic), stabilisers (e.g. carrageenan, alginate), preservatives (e.g. benzoic acid and its salts, sorbic acid and its salts), antioxidants (e.g. tocopherol, ascorbic acid), chelators (e.g. citric acid), organic or inorganic acidifying agents (e.g. acetic acid, phosphoric acid), additional bitter substances (e.g. quinine, caffeine, limonene, amarogentine, humulone, lupulone, catechols, tannins), substances that prevent enzymatic browning (e.g. sulfite, ascorbic acid), ethereal oils, plant extracts, natural or synthetic colourings or colouring pigments (e.g. carotinoids, flavonoids, anthocyans, chlorophyll and derivatives thereof), spices, trigeminally active substances or plant extracts containing such trigeminally active substances, synthetic, natural or nature-identical flavourings or odorants as well as odour correctors.

Orally consumable products (in particular foodstuffs, feeds and medicaments) according to the invention, for example those in the form of preparations or semi-finished products, preferably comprise a flavour composition in order to complete and refine the taste and/or odour. A preparation can comprise as constituents a solid carrier and a flavour composition. Suitable flavour compositions comprise, for example, synthetic, natural or nature-identical flavourings, odorants and taste-imparting substances, reaction flavourings, smoke flavourings or other flavour-giving preparations (e.g. protein (partial) hydrolysates, preferably protein (partial) hydrolysates having a high arginine content, barbecue flavourings, plant extracts, spices, spice preparations, vegetables and/or vegetable preparations) as well as suitable auxiliary substances and carriers. Particularly suitable here are the flavour compositions or constituents thereof which produce a roasted, meaty (in particular chicken, fish, seafood, beef, pork, lamb, mutton, goat), vegetable-like (in particular tomato, onion, garlic, celery, leek, mushroom, aubergine, seaweed), spicy (in particular black and white pepper, cardamom, nutmeg, pimento, mustard and mustard products), fried, yeast-like, boiled, fatty, salty and/or pungent flavour impression and accordingly can enhance the spicy impression. The flavour compositions generally comprise more than one of the mentioned ingredients.

The energy density of an orally consumable product (in particular foodstuff, feed or medicament) can be lowered by replacing energy-rich ingredients of the orally consumable product with substitute material's (e.g. low-calorie thickeners instead of fats, low-calorie or calorie-free sweeteners instead of conventional sugars). The disadvantage already discussed above, that the consumer consumes an orally consumable product (in particular a foodstuff) having a reduced energy density in larger amounts and, in the worst case, the intake of calorically relevant food constituents is then even increased, is counteracted by the use of N-nonanoylvanillylamine in orally consumable foodstuffs (in particular in foodstuffs). N-Nonanoylvanillylamine contained in an orally consumable product imparts a premature feeling of fullness and at the same time reduces the appetite. In addition, it has a positive influence on the mood of the consumer, which likewise has a positive effect.

An orally consumable product according to the invention is preferably selected from the group comprising
confectionery, preferably reduced-calorie or calorie-free confectionery, preferably selected from the group comprising muesli bar products, fruit gums, dragées, hard caramels and chewing gum,
non-alcoholic drinks, preferably selected from the group comprising green tea, black tea, (green, black) tea drinks enriched with (green, black) tea extracts, rooibos tea, other herbal teas, fruit-containing low-sugar or sugar-free soft drinks, isotonic drinks, nectars, fruit and vegetable juices, fruit and vegetable juice preparations,
instant drinks, preferably selected from the group comprising instant (green, black, rooibos, herbal) tea drinks,
cereal products, preferably selected from the group comprising low-sugar and sugar-free breakfast cereals and muesli bars,
dairy products, preferably selected from the group comprising reduced-fat and fat-free milk drinks, yoghurt, kefir, whey, buttermilk and ice-cream,
products made from soy protein or other soybean fractions, preferably selected from the group comprising soy milk, products produced from soy milk, drinks containing isolated or enzymatically treated soy protein, drinks containing soy flour, preparations containing soy lecithin, products produced from preparations containing soy lecithin and mixtures with fruit preparations and optionally flavours,
sweetener preparations, tablets and sachets,
sugar-free dragées,
ice-cream, with or without milk-based constituents, preferably sugar-free.

An orally consumable product (in particular foodstuff, feed or medicament) according to the invention preferably comprises (a) one, two or more sweeteners and/or (b) one, two or more thickeners.

The term “sweeteners” here denotes substances having a relative sweetening power of at least 25, based on the sweetening power of sucrose (which accordingly has a sweetening power of 1). Sweeteners to be used in an orally consumable product (in particular foodstuff, feed or medicament) according to the invention (a) are preferably non-cariogenic and/or have an energy content of not more than 5 kcal per gram of the orally consumable product.

Advantageous sweeteners in a preferred orally consumable product (in particular foodstuff, feed or medicament) according to the invention are selected from the following groups (a1)) and (a2):
(a1)) naturally occurring sweeteners, preferably selected from the group comprising
(a1-1) miraculin, monellin, mabinlin, thaumatin, curculin, brazzein, pentaidin, D-phenylalanine, D-tryptophan, and extracts or fractions obtained from natural sources, comprising those amino acids and/or proteins, and the physiologically acceptable salts of those amino acids and/or proteins, in particular the sodium, potassium, calcium or ammonium salts;
(a1-2) neohesperidin dihydrochalcone, naringin dihydrochalcone, stevioside, steviolbioside, rebaudiosides, in particular rebaudioside A, rebaudioside B, rebaudioside C, rebaudioside D, rebaudioside E, rebaudioside F, rebaudioside G, rebaudioside H, dulcosides and rubusoside, suavioside A, suavioside B, suavioside G, suavioside H, suavioside I, suavioside J, baiyunoside 1, baiyunoside 2, phlomisoside 1, phlomisoside 2, phlomisoside 3 and phlomisoside 4, abrusoside A, abrusoside B, abrusoside C, abrusoside D, cyclocaryoside A and cyclocaryoside 1, osladin, polypodoside A, strogin 1, strogin 2, strogin 4, selligueain A, dihydroquercetin 3-acetate, perillartin, telosmoside A₁₅, periandrin I-V, pterocaryosides, cyclocaryosides, mukuroziocides, trans-anethole, trans-cinnamaldehyde, bryosides, bryonosides, bryonodulcosides, camosiflosides, scandenosides, gypenosides, trilobatin, phloridzin, dihydroflavanols, hematoxylin, cyanin, chlorogenic acid, albiziasaponin, telosmosides, gaudichaudioside, mogrosides, mogroside V, hernandulcins, monatin, phyllodutcin, glycyrrhetinic acid and derivatives thereof, in particular glycosides thereof such as glycyrrhizine, and the physiologically acceptable salts of those compounds, in particular the sodium, potassium, calcium or ammonium salts;
(a1-3) extracts or concentrated fractions of the extracts, selected from the group comprising thaumatococcus extracts (katamfe plant), extracts from Stevia ssp. (in particular Stevia rebaudiana), swingle extracts (Momordica or Siratia grosvenorii, Luo-Han-Guo), extracts from Glycerrhyzia ssp. (in particular Glycerrhyzia glabra), extracts from Rubus ssp. (in particular Rubus suavissimus), citrus extracts and extracts from Lippia dulcis;
(a2) synthetic sweet-tasting substances, preferably selected from the group comprising magap, sodium cyclamate or other physiologically acceptable salts of cyclamic acid, acesulfame K or other physiologically acceptable salts of acesulfame, neohesperidin dihydrochalcone, naringin dihydrochalcone, saccharin, saccharin sodium salt, aspartame, superaspartame, neotame, alitame, advantame, perillartin, sucralose, lugduname, carrelame, sucrononate and sucrooctate.

Advantageous thickeners in a preferred orally consumable product (in particular foodstuff, feed or medicament) according to the invention are selected from the group comprising: crosslinked polyacrylic acids and derivatives thereof, polysaccharides and derivatives thereof, such as xanthan gum, agar-agar, alginates or tyloses, cellulose derivatives, for example carboxymethylcellulose or hydroxycarboxymethylcellulose, fatty alcohols, monoglycerides and fatty acids, polyvinyl alcohol and polyvinylpyrrolidone.

Preference is given according to the invention to an orally consumable product (in particular foodstuff or feed) which comprises milk thickened with lactic acid bacteria and/or cream thickened with lactic acid bacteria and which preferably

is selected from the group comprising orally consumable products having a fat content of 4.0 wt. % or less, preferably of 1.5 wt. % or less, particularly preferably 0.5 wt. % or less, in each case based on the total weight of the orally consumable product,

and/or

is selected from the group comprising yoghurt, kefir and quark.

The orally consumable product (in particular foodstuff or feed) according to the invention comprising milk thickened with lactic acid bacteria and/or cream thickened with lactic acid bacteria preferably has an energy content of not more than 150 kcal/100 g of the orally consumable product, preferably not more than 100 kcal/100 g, particularly preferably not more than 75 kcal/100 g, particularly preferably not more than 50 kcal/100 g.

A preferred orally consumable product (in particular foodstuff or feed) according to the invention comprising milk thickened with lactic acid bacteria and/or cream thickened with lactic acid bacteria additionally comprises fruits and/or fruit preparations.

Particular preference is given to an orally consumable product (in particular foodstuff or feed) according to the invention comprising milk thickened with lactic acid bacteria and/or cream thickened with lactic acid bacteria, wherein the orally consumable product comprises (i) sugars and/or (ii) thickeners and/or (iii) gelling agents and/or (iv) sweeteners and/or (v) flavours and/or (vi) preservatives.

“Sugar” within the context of the present text (unless indicated otherwise or otherwise apparent from the context) is the collective term for all sweet-tasting saccharides (single and double sugars).

An orally consumable product (in particular foodstuff or feed) according to the invention comprising milk thickened with lactic acid bacteria and/or cream thickened with lactic acid bacteria is advantageously an orally consumable product which comprises a probiotic, wherein the probiotic is preferably selected from the group comprising Bifidobacterium animalis subsp. lactis BB-12, Bifidobacterium animalis subsp. lactis DN-173 010, Bifidobacterium animalis subsp. lactis HNO19, Lactobacillus acidophilus LA5, Lactobacillus acidophilus NCFM, Lactobacillus johnsonii La1, Lactobacillus casei immunitass/defensis, Lactobacillus casei Shirota (DSM 20312), Lactobacillus casei CRL431, Lactobacillus reuteri (ATCC 55730) and Lactobacillus rhamnosus (ATCC 53013).

Particular preference is given to an orally consumable product (in particular foodstuff, feed or medicament) according to the invention that is a chewing gum and comprises a chewing-gum base. The chewing-gum base is preferably selected from the group comprising chewing-gum or bubble-gum bases. The latter are softer, so that gum bubbles can also be formed therewith. Preferred chewing-gum bases according to the invention include, in addition to the natural resins or the natural latex chicle that are traditionally used, elastomers such as polyvinyl acetate (PVA), polyethylene, (low or medium molecular weight) polyisobutene (PIB), polybutadiene, isobutene-isoprene copolymers (butyl rubber), polyvinyethyl ether (PVE), polyvinylbutyl ether, copolymers of vinyl esters and vinyl ethers, styrene-butadiene copolymers (styrene-butadiene rubber, SBR) or vinyl elastomers, for example based on vinyl acetate/vinyl laurate, vinyl acetate/vinyl stearate or ethylene/vinyl acetate, as well as mixtures of the mentioned elastomers, as described, for example, in EP 0 242 325, U.S. Pat. No. 4,518,615, U.S. Pat. No. 5,093,136, U.S. Pat. No. 5,266,336, U.S. Pat. No. 5,601,858 or U.S. Pat. No. 6,986,709. In addition, chewing-gum bases that are preferably to be used according to the invention preferably comprise further constituents such as, for example, (mineral) fillers, plasticisers, emulsifiers, antioxidants, waxes, fats or fatty oils, such as, for example, hardened (hydrogenated) vegetable or animal fats, mono-, di- or tri-glycerides. Suitable (mineral) fillers are, for example, calcium carbonate, titanium dioxide, silicon dioxide, talcum, aluminium oxide, dicalcium phosphate, tricalcium phosphate, magnesium hydroxide and mixtures thereof. Suitable plasticisers, or agents for preventing adhesion (detackifiers), are, for example, lanolin, stearic acid, sodium stearate, ethyl acetate, diacetin (glycerol diacetate), triacetin (glycerol triacetate), triethyl citrate. Suitable waxes are, for example, paraffin waxes, candelilla wax, carnauba wax, microcrystalline waxes and polyethylene waxes. Suitable emulsifiers are, for example, phosphatides such as lecithin, mono- and di-glycerides of fatty acids, for example glycerol monostearate.

Chewing gums according to the invention (in particular as disclosed above) preferably comprise constituents such as sugars of different types, sugar substitutes, other sweet-tasting substances, sugar alcohols (in particular sorbitol, xylitol, mannitol), ingredients having a cooling effect, taste correctors for unpleasant taste impressions, further taste-modulating substances (e.g. inositol phosphate, nucleotides such as guanosine monophosphate, adenosine monophosphate or other substances such as sodium glutamate or 2-phenoxypropionic acid), humectants, thickeners, emulsifiers, stabilisers, odour correctors and flavours (e.g. eucalyptus-menthol, cherry, strawberry, grapefruit, vanilla, banana, citrus, peach, blackcurrant, tropical fruits, ginger, coffee, cinnamon, combinations (of the mentioned flavours) with mint flavours as well as spearmint and peppermint on their own). The combination inter alia of the flavours with further substances that have cooling, warming and/or mouth-watering properties is of particular interest.

Particular preference is given to an orally consumable product (in particular foodstuff, feed or medicament) according to the invention, wherein the orally consumable product is a drink,

wherein the drink preferably has a sugar content of 30 g/100 ml of drink or less, preferably of 15 g/100 ml or less, particularly preferably 5 g/100 ml or less, particularly preferably contains no sugar,

and/or

wherein the drink contains no ethanol or contains not more than 0.1 percent by volume ethanol, based on the volume of the drink.

Within the context of the present invention, orally consumable products according to the invention which are ethanol-containing drinks are less preferred.

In the present invention, no ethanol means that no ethanol is added and that the preparation comprises less than 0.1 vol %, preferably less than 0.01 vol % and particularly preferably no measurable amount of ethanol.

Particular preference is given to orally consumable products (preferably foodstuffs, feeds or medicaments) according to the invention, wherein the product in question is a carbonated drink or an uncarbonated drink.

Our own investigations have shown that neither the pungent taste nor the action of N-nonanoylvanillylamine on the pungency receptor TRPV1 is necessary for the actions according to the invention (appetite reduction; fullness; mood enhancement). Therefore, in preferred orally consumable products (in particular foodstuffs, feeds or medicaments), the N-nonanoylvanillylamine is combined with a further flavouring, foodstuff, feed or medicament constituent that reduces or completely eliminates the TRPV1 response, that is to say with a TRPV1 inhibitor, preferably a TRPV1 inhibitor permitted as a flavouring.

Flavourings, foodstuff, feed or medicament constituents which act as TRPV1 inhibitors can be, for example: para-tert-butylcyclohexanol according to WO 2009 087,242, eriodictyol according to Rossato, M. F.; Trevisan, G.; Walker, C. I. B.; Klafke, J. Z.; de Oliveira, A. P.; Villarinho, J. G.; Zanon, R. B.; Royes, L. F. F.; Athayde, M. L.; Gomez, M. V.; Ferreira, J., Eriodictyol: A flavonoid antagonist of the TRPV1 receptor with antioxidant activity. Biochemical Pharmacology 2011, 814, (544-551), or eriodictyol-containing plant products such as, for example, an extract from Eriodictyon ssp. or Citrus ssp.

For the release of dopamine by N-nonanoylvanillylamine, this is shown by way of example in Example 2 below; it is clear therefrom that the release of dopamine is not dependent on the presence of a TRPV1 inhibitor, in this case para-tert-butylcyclohexanol.

The invention relates also to a substance mixture according to the invention or an orally consumable product (in particular foodstuff, feed or medicament) according to the invention additionally comprising one or more TRPV1 inhibitors, preferably selected from the group comprising trans-tert-butylcyclohexanol and eriodictyol.

In a further embodiment of the present invention, N-nonanoylvanillylamine is used in combination with at least one substance for masking or reducing an unpleasant (bitter, metallic, chalky, acidic, astringent, pungent) taste impression or for enhancing or producing a pleasant taste impression (sweet, salty, umami).

An enhancement of the taste can be achieved in that manner. These further substances can be selected from the following list, without thereby limiting the invention: trans-tert-butylcyclohexanol according to WO 2009 087,242, monosodium glutamate, glutamic acid, nucleotides (e.g. adenosine 5′-monophosphate, cytidine 5′-monophosphate, inosine 5′-monophosphate, guanosine 5′-monophosphate) or pharmaceutically acceptable salts thereof, lactisols, hydroxyflavanones (e.g. eriodictyol, homoeriodictyol or the sodium salts thereof), in particular according to EP 1 258 200, hydroxybenzoic acid amides (e.g. 2,4-dihydroxybenzoic acid vanillylamide, 4-hydroxybenzoic acid vanillylamide), mixtures of whey proteins with lecithins, yeast extracts, plant hydrolysates, powdered vegetables (e.g. onion powder, tomato powder), plant extracts (e.g. of lovage or mushrooms such as shiitake), marine algae and mineral salt mixtures as well as mixtures according to WO 2007/045,566.

In a preferred embodiment of the present invention, N-nonanoylvanillylamine is used in the reduced-calorie compositions, preparations and semi-finished products according to the invention in combination with at least one sweetness-enhancing substance, in particular with one or more compounds according to WO 2007/104879 A1 or WO 2007/107596 A1, especially together with hesperetin and/or phioretin. The taste profile is thereby enhanced and deepened as well as completed. The total content of hesperetin and/or phloretin in such compositions or preparations is preferably in the range of from 1 to 400 ppm, preferably in the range of from 5 to 200 ppm, based on the total weight of the composition or preparation.

In addition to one or more sweetness-enhancing substances, the compositions, preparations and semi-finished products according to the invention can preferably comprise taste-imparting substances which bring about a tingling or cooling effect. Accordingly, when N-nonanoylvanillylamine was combined with hesperetin and/or phloretin on the one hand and with cis- and/or trans-pellitorin (see WO 2004/000787 or WO 2004/043906) on the other hand, a further improved taste profile was achieved which was preferred by consumers. The total content of cis- and/or trans-pellitorin in such compositions or preparations is preferably in the range of from 0.5 to 500 ppm, preferably in the range of from 5 to 100 ppm, based on the total weight of the composition or preparation.

Modulating flavourings and/or taste-imparting substances are preferably selected from the group comprising trans-tert-butylcyclohexanol, adenosine 5′-monophosphate, cytidine 5′-monophosphate, inosine 5′-monophosphate and the pharmaceutically acceptable salts thereof; Iactisols; 2,4-dihydroxybenzoic acid; 3-hydroxybenzoic acid; sodium salts, preferably sodium chloride, sodium lactate, sodium citrate, sodium acetate, sodium gluconoate; hydroxyflavanones, such as, for example, eriodictyol, homoeriodictyol and the sodium salts thereof; hydroxybenzoic acid amides, such as, for example, 2,4-dihydroxybenzoic acid vanillylamide, 2,4-dihydroxybenzoic acid N-(4-hydroxy-3-methoxybenzyl)amide, 2,4,6-trihydroxybenzoic acid N-(4-hydroxy-3-methoxybenzyl)amide, 2-hydroxybenzoic acid N-4-(hydroxy-3-methoxybenzyl)amide, 4-hydroxybenzoic acid N-(4-hydroxy-3-methoxybenzyl)amide, 2,4-dihydroxybenzoic acid N-(4-hydroxy-3-methoxybenzyl)amide monosodium salt, 2,4-dihydroxybenzoic acid N-2-(4-hydroxy-3-methoxyphenyl)-ethyl-amide, 2,4-dihydroxybenzoic acid N-(4-hydroxy-3-ethoxybenzyl)amide, 2,4-dihydroxybenzoic acid N-(3,4-dihydroxybenzyl)amide and 2-hydroxy-5-methoxy-N-[2-(4-hydroxy-3-methoxyphenyl)ethyl)amide; 4-hydroxybenzoic acid vanillylamide (in particular those as described in WO 2006/024587 which, in respect of the corresponding compounds disclosed therein, is incorporated by reference into this application); hydroxydeoxybenzoins, such as, for example, 2-(4-hydroxy-3-methoxyphenyl)-1-(2,4,6-trihydroxyphenyl)ethanone, 1-(2,4-dihydroxyphenyl)-2-(4-hydroxy-3-methoxy-phenyl)ethanone, 1-(2-hydroxy-4-methoxyphenyI)-2-(4-hydroxy-3-methoxyphenyl)ethanone) (in particular those as described in WO 2006/106023 which, in respect of the corresponding compounds disclosed therein, is incorporated by reference into this application); hydroxyphenylalkanediones, such as, for example, gingerdione-[2], gingerdione-[3], gingerdione-[4], dehydrogingerdione-[2], dehydrogingerdione-[3], dehydrogingerdione-[4]) (in particular those as described in WO 2007/003527 which, in respect of the corresponding compounds disclosed therein, is incorporated by reference into this application); diacetyl trimers (in particular those as described in WO 2006/058893 which, in respect of the corresponding compounds disclosed therein, is incorporated by reference into this application); gamma-aminobutyric acids (in particular those as described in WO 2005/096841 which, in respect of the corresponding compounds disclosed therein, is incorporated by reference into this application) and divanillins (in particular divanillin as described in WO 2004/078302 which, in respect of the corresponding compounds disclosed therein, is incorporated by reference into this application); bicyclo[4.1.0]heptane-7-carboxylic acid amides, in particular those as described in EP 2 079 322 (Symrise) which, in respect of the corresponding compounds disclosed therein, is incorporated by reference into this application; zo cyclopropanecarboxylic acid (3-methylcyclohexyl)amides, in particular those as described in EP 1 989 944 (Symrise) which, in respect of the corresponding compounds disclosed therein, is incorporated by reference into this application; aromatic neo-menthylamides, in particular those as described in EP 2 064 959 (Symrise) which, in respect of the corresponding compounds disclosed therein, is incorporated by reference into this application); neo-menthylamides, in particular those as described in US 2009/311401 A (Symrise) which, in respect of the corresponding compounds disclosed therein, is incorporated by reference into this application.

Further aspects of the present invention will become apparent from the following examples and the accompanying claims.

### DESCRIPTION OF THE FIGURES

FIG. 1 shows the release of dopamine by SH-SY5Y cells following stimulation with 100 μM acetylcholine (positive control) or different concentrations of N-nonanoylvanillylamine (nonivamide). The results are shown normalised to the control in % (TIC [%]). n=6 (3 biological and 2 technical replicates); *p≦0.05 vs. control **p≦0.01 vs. control ***p≦0.001 vs. control. The letters used in the figure mean: A=control (buffer) B=EtOH control (buffer 0.1% EtOH) C=acetylcholine 100 μM, D=N-nonanoylvanillylamine 0.1 μM, 1 μM, or 10 μM. The data relating to FIG. 1 are shown in table form below:

| | | Standard |
|---|---|---|
| Test substance | T/C [%] | deviation |
| Control | 100 | 25.72 |
| Ethanol control | 100 | 5.99 |
| Acetylcholine 100 μM (positive control) | 139.84 | 32.91 |
| N-Nonanoylvanillylamine 0.1 μM | 697.84 | 145.14 |
| N-Nonanoylvanillylamine 1 μM | 1350.94 | 775.88 |
| N-Nonanoylvanillylamine 10 μM | 1774.98 | 290.45 |

FIG. 2 shows the release of dopamine by SH-SY5Y cells following stimulation with 1 μM N-nonanoylvanillylamine(nonivamide) or with 1 μM N-nonanoylvanillylamine (nonivamide) and different concentrations of the selective TRPV1 inhibitor trans-4-tert-butylcyclohexanol. The results are shown normalised to the control in % (TIC [%]). n=4 (2 biological and 2 technical replicates). Significant differences between the treatments were determined by means of Student's T test and are shown by the letters a, b and c. The letters used in the figure mean: A=control (buffer), B=N-nonanoylvanillylamine 1 μM, C=25 μM trans-4-tert-butylcyclohexanol, 50 μM trans-4-tert-butylcyclohexanol, 80 μM trans-4-tert-butylcyclohexanol, C+B=trans-4-tert-butylcyclohexanol+1 μM N-nonanoylvanillylamine.

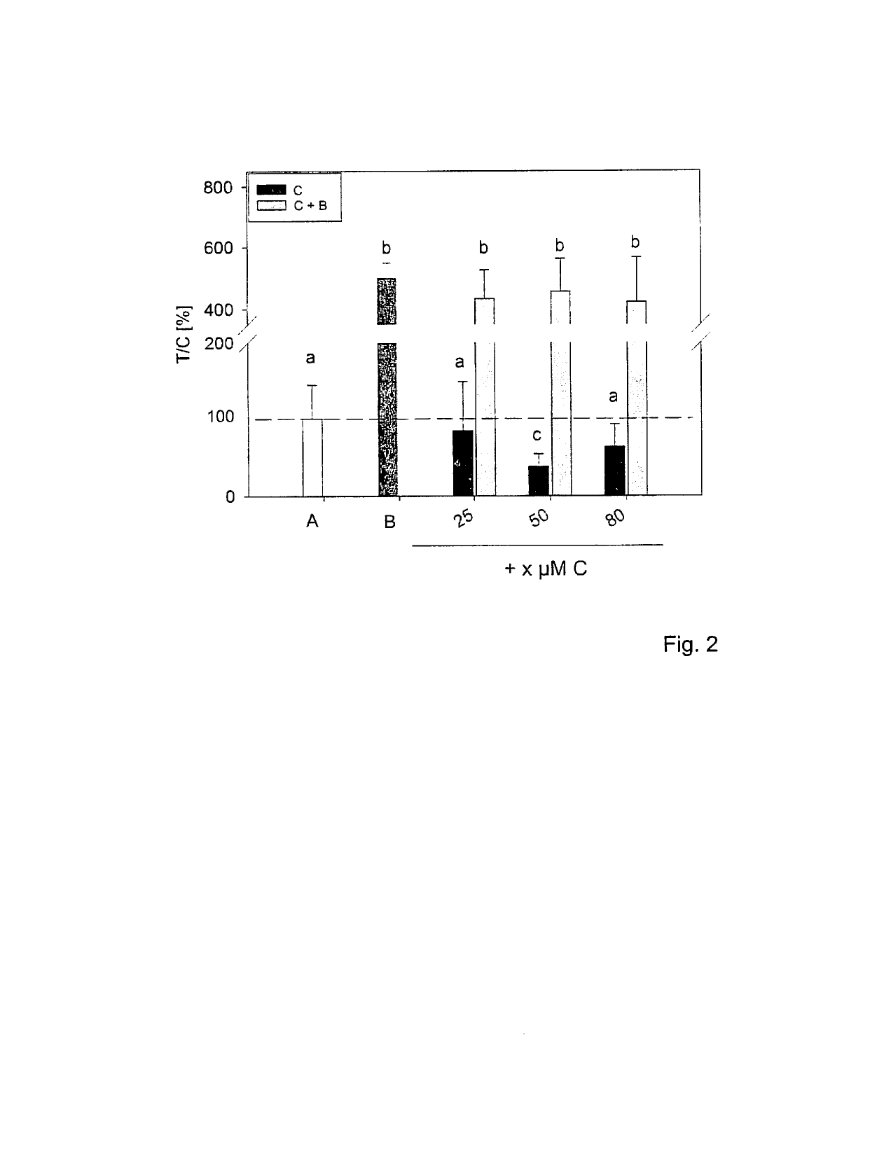

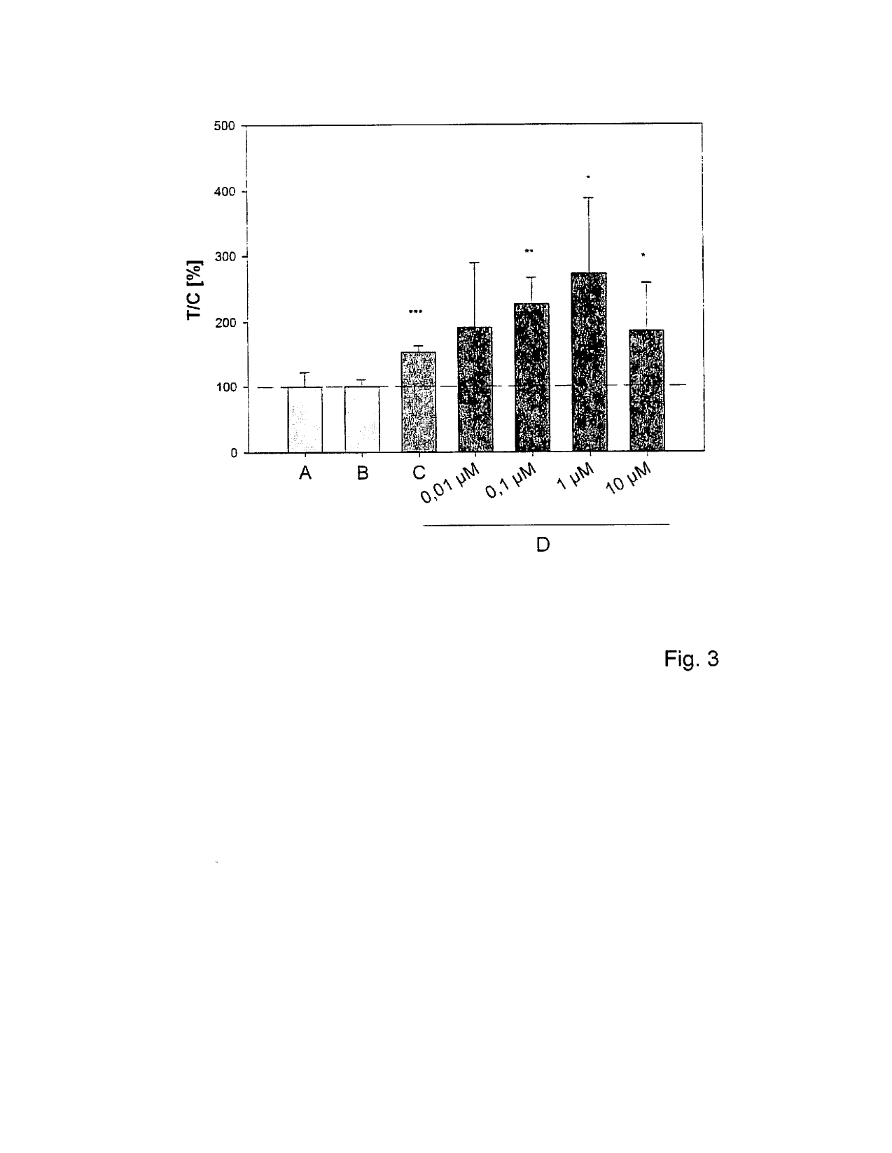

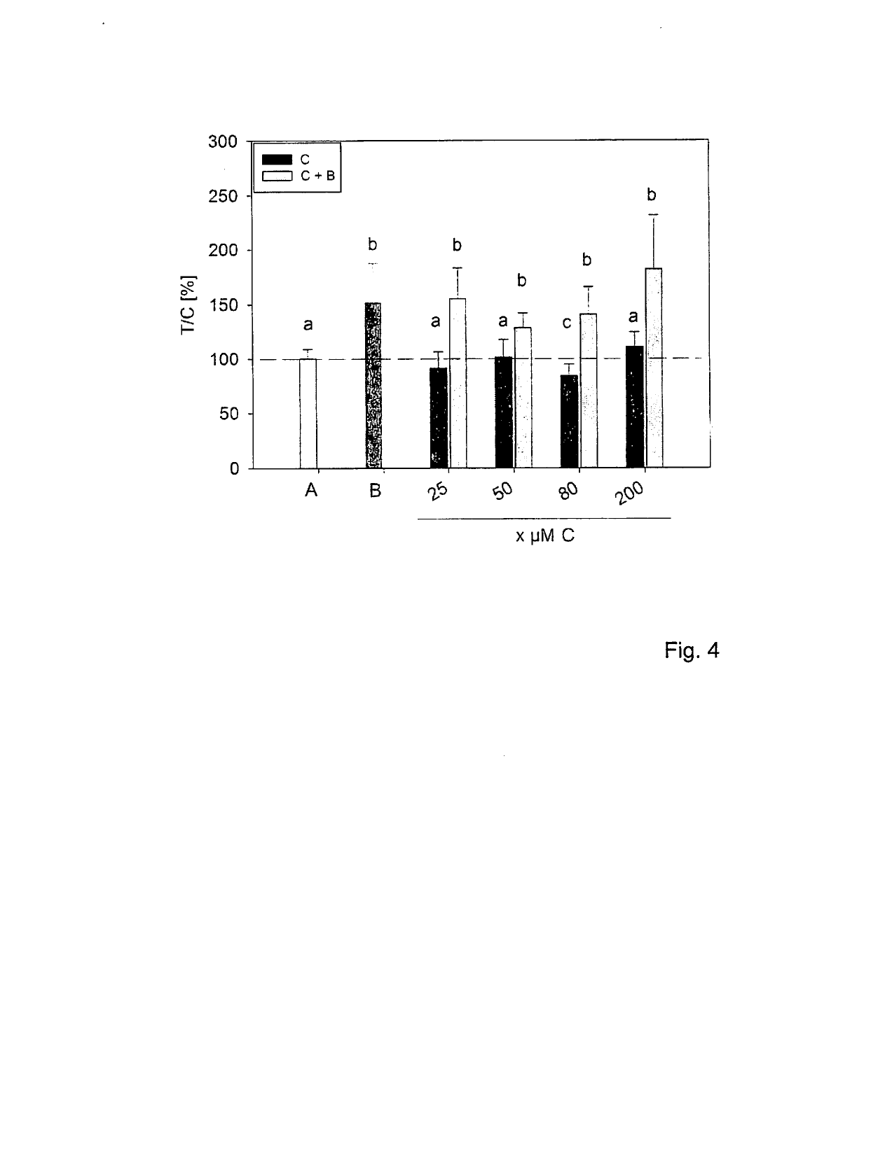

The data relating to FIG. 2 are shown in table form below:

| | | Standard |
|---|---|---|
| Test substance | T/C [%] | deviation |
| Control | 100 | 44.44 |
| N-Nonanoylvanillylamine 1 μM | 501.20 | 47.86 |
| N-Nonanoylvanillylamine 1 μM + 25 μM | 433.55 | 92.86 |
| trans-tert-butylcyclohexanol | | |
| N-Nonanoylvanillylamine 1 μM + 50 μM | 456.80 | 105.52 |
| trans-4-tert-butylcyclohexanol | | |
| N-Nonanoylvanillylamine 1 μM + 80 μM | 422.48 | 143.41 |
| trans-4-tert-butylcyclohexanol | | |
| 25 μM trans-4-tert-butylcyclohexanol | 84.47 | 63.77 |
| 50 μM trans-4-tert-butylcyclohexanol | 38.19 | 16.06 |
| 80 μM trans-4-tert-butylcyclohexanol | 63.53 | 29.42 |

FIG. 3 shows the release of serotonin by SH-SY5Y cells following stimulation with 50 mM potassium chloride (50 mM KCl, positive control) or different concentrations of N-nonanoylvanillylamine. The results are shown normalised to the control in % (TIC [%]). n=6 (3 biological and 2 technical replicates); *p≦0.05 vs. control **p≦0.01 vs. control ***p≦0.001 vs. control. The letters used in the figure mean: A=control (buffer), B=EtOH (buffer 0.1% EtOH), C=50 nM KCl, D=N-nonanoylvanillylamine 0.01 μM, 0.1 μM, 1 μM or 10 μM.

The data relating to FIG. 3 are shown in table form below:

| | | Standard |
|---|---|---|
| Test substance | T/C [%] | deviation |
| Control | 100.00 | 22.31 |
| Ethanol control | 100.00 | 10.80 |
| 50 mM KCl (positive control) | 153.48 | 9.9 |
| N-Nonanoylvanillylamine 0.01 μM | 191.35 | 98.18 |
| N-Nonanoylvanillylamine 0.1 μM | 226.44 | 39.71 |
| N-Nonanoylvanillylamine 1 μM | 272.12 | 115.53 |
| N-Nonanoylvanillylamine 10 μM | 185.65 | 72.76 |

FIG. 4 shows the release of serotonin by SH-SY5Y cells following stimulation with 1 μM N-nonylvanillylamine(nonivamide) or with 1 μM N-nonanoylvanillylamine (nonivamide) and different concentrations of the selective TRPV1 inhibitor trans-4-tert-butylcyclohexanol. The results are shown normalised to the control in % (TIC [%]). n=4 (2 biological and 2 technical replicates). Significant differences between the treatments were determined by means of Student's T test and are shown by the letters a, b and c. The letters used in the figure mean: A=control (buffer), B=N-nonanoylvanillylamine 1 μM, C=25 μM 50 μM 80 μM trans-4-tert-butylcyclohexanol, or 200 μM trans-4-tert-butylcyclohexanol, C+B=trans-4-tert-butylcyclohexanol+1 μM N-nonanoylvanillylamine.

The data relating to FIG. 4 are shown in table form below:

| | | Standard |
|---|---|---|
| Test substance | T/C [%] | deviation |
| Control | 100.00 | 8.97 |
| N-Nonanoylvanillylamine 1 μM | 151.75 | 35.46 |
| N-Nonanoylvanillylamine 1 μM + 25 μM | 154.86 | 28.13 |
| trans-tert-butylcyclohexanol | | |
| N-Nonanoylvanillylamine 1μM + 50 μM | 128.74 | 13.12 |
| trans-tert-butylcyclohexanol | | |
| N-Nonanoylvanillylamine 1 μM + 80 μM | 140.81 | 24.92 |
| trans-tert-butylcyclohexanol | | |
| N-Nonanoylvanillylamine 1 μM + 200 μM | 181.93 | 49.64 |
| trans-tert-butylcyclohexanol | | |
| 25 μM trans-tert-butylcyclohexanol | 91.07 | 15.20 |
| 50 μM trans-tert-butylcyclohexanol | 101.39 | 16.59 |
| 80 μM trans-tert-butylcyclohexanol | 84.129 | 10.73 |
| 200 μM trans-tert-butylcyclohexanol | 110.99 | 13.76 |

### EXAMPLES

Human neuroblastoma cells (SH-SY5Y, ATCC number CRL-2266) are used as the cell model. Cultivation takes place at 37° C. and 5% CO₂ content with a mixture consisting of equal parts of Eagle's Minimum Essential Medium (MEM) and F12 medium (in each case with 10% FBS and 1% penicillin/streptomycin). For measurement of the release of dopamine and serotonin, the cells are harvested with trypsin and, after a vitality test by trypan blue staining, are sown in a defined cell number in 35 mm cell culture dishes.

### Example 1

### Release of Dopamine in an Experimental Cell System by Means of N-Nonanovlvanillvlamine

Method for measuring the release of dopamine:

After stimulation of 1.25*10⁶ human neuroblastoma cells (SH-SY5Y) for 3 minutes with 350 μl of Krebs-Ringer HEPES buffer, pH 5, with or without addition of N-nonanoylvanillylamine (wherein in the case of addition of N-nonanoylvanillylamine, concentrations of 0.1 μM, 1 μM and 10 μM are established in the Krebs-Ringer HEPES buffer), the supernatant is acidified with 1 N HCl and the dopamine content is determined by means of an enzyme-based detection method (dopamine ELISA, DLD Diagnostica, Hamburg, Germany). The cells are lysed with a buffer containing sodium lauryl sarcosinate, and the DNA content is determined by means of a NanoQuant plate (Tecan, Ménnendorf, Switzerland) for normalisation of the dopamine release. In the case of an ethanol control (EtOH control), no N-nonanoylvanillylamine is added to the Krebs-Ringer HEPES buffer, but ethanol is added to the buffer so that a 0.1% ethanolic Krebs-Ringer HEPES buffer solution is obtained. In the case of the positive control, acetylcholine is used instead of the N-nonanoylvanillylamine, a concentration of 100 μM acetylcholine being established in the Krebs-Ringer HEPES buffer.

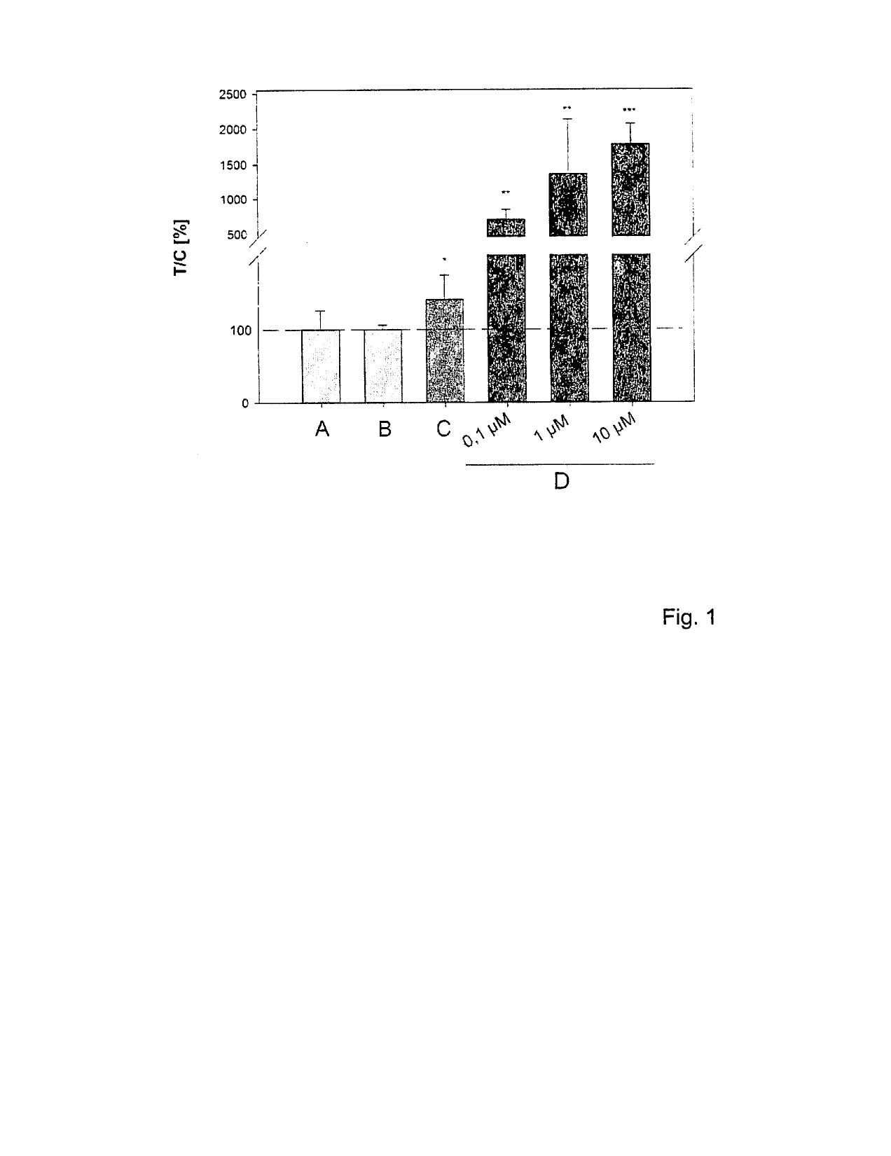

TABLE 1
Data relating to FIG. 1 (release of dopamine by SH-SY5Y cells following
stimulation with 100 μM acetylcholine (positive control)
or different concentrations of N-nonanoylvanillylamine (nonivamide)).

| Test substance | T/C [%] | Standard deviation |
|---|---|---|
| Control | 100 | 25.72 |
| EtOH control | 100 | 5.99 |
| Acetylcholine 100 μM | 139.84 | 32.91 |
| N-Nonanoylvanillylamine 0.1 μM | 697.84 | 145.14 |
| N-Nonanoylvanillylamine 1 μM | 1350.94 | 775.88 |
| N-Nonanoylvanillylamine 10 μM | 1774.98 | 290.45 |

FIG. 1 shows the very considerable influence of small concentrations of N-nonanoylvanillylamine on the release of dopamine. A concentration of only 100 nM of N-nonanoylvanillylamine leads to a seven-fold increase in the amount of dopamine released by SH-SY5Y cells.

### Example 2

### Release of Dopamine in an Experimental Cell System with N-nonanovivanillylamine and a TRPV1 Inhibitor

After stimulation of 1.25*10⁶ human neuroblastoma cells (SH-SY5Y) for 3 minutes with 350 μl of Krebs-Ringer HEPES buffer, pH 5, with or without addition of 1 μM N-nonanolyvanillylamine (wherein in the case of addition of N-nonanoylvanillylamine, a concentration of 1 μM is established in the Krebs-Ringer HEPES buffer) in combination with different application-relevant amounts of the selective TRPV1 inhibitor trans-4-tert-butylcyclohexanol (so that concentrations of 25, 50 and 80 μM of trans-4-tert-butylcyclo-hexanol are established in the Krebs-Ringer HEPES buffer), the supernatant is acidified with 1 N HCl and the dopamine content is determined by means of an enzyme-based detection method (dopamine ELISA, DLD Diagnostica, Hamburg, Germany). The cells are lysed with a buffer containing sodium lauryl sarcosinate, and the DNA content is determined by means of a NanoQuant plate (Tecan, Ménnendorf, Switzerland) for normalisation of the dopamine release. For comparison purposes, the tests were repeated without the addition of N-nonanoylvanillylamine.

TABLE 2
Release of dopamine by SH-SY5Y cells following stimulation with 1 μM
N-nonylvanillylamine (nonivamide) and different concentrations of
the selective TRPV1 inhibitor trans-4-tert-butylcyclohexanol

| Test substance | T/C [%] | Standard deviation |
|---|---|---|
| Control | 100.0 | 44.44 |
| N-Nonanoylvanillylamine 1 μM | 501.20 | 47.86 |
| N-Nonanoylvanillylamine 1 μM + 25 μM | 433.55 | 92.86 |
| trans-4-tert-butylcyclohexanol | | |
| N-Nonanoylvanillylamine 1 μM + 50 μM | 456.80 | 105.52 |
| trans-4-tert-butylcyclohexanol | | |
| N-Nonanoylvanillylamine 1μM + 80 μM | 422.48 | 143.41 |
| trans-4-tert-butylcyclohexanol | | |
| 25 μM trans-4-tert-butylcyclohexanol | 84.47 | 63.77 |
| 50 μM trans-4-tert-butylcyclohexanol | 38.19 | 16.06 |
| 80 μM trans-4-tert-butylcyclohexanol | 63.53 | 29.42 |

The five-fold increase in the amount of dopamine released as a result of 1 μM N-nonanoylvanillylamine was not significantly influenced by simultaneous stimulation with the selective TRPV1 inhibitor trans-4-tert-butylcyclohexanol in the application-relevant concentrations (25, 50 and 80 μM). The data accordingly show that the TRPV1 receptor, which is responsible for the pungent taste, is not necessary for the release of dopamine increased by N-nonanoylvanillylamine.

### Example 3

### Release of Serotonin in an Experimental Cell System with N-nonanoylvanillylamine

After stimulation of 1.25*10⁶ human neuroblastoma cells (SH-SY5Y) for 3 minutes with 300 μl of Krebs-Ringer HEPES buffer, 0.1% ascorbic acid, pH 6.2, with or without addition of N-nonanoylvanillylamine (wherein in the case of addition of N-nonanoylvanillylamine, concentrations of 0.01 μM, 0.1 μM, 1 μM and 10 μM are established in the Krebs-Ringer HEPES buffer), the serotonin content is determined by means of an enzyme-based detection method (serotonin ELISA sensitive, QLD Diagnostica, Hamburg, Germany). The cells are lysed with a buffer containing sodium lauryl sarcosinate, and the DNA content is determined by means of a NanoQuant plate (Tecan, Ménnendorf, Switzerland) for normalisation of the serotonin release.

TABLE 3
Release of serotonin by SH-SY5Y cells following stimulation with
50 mM potassium chloride (50 mM KCl, positive control) or
different concentrations of N-nonanoylvanillylamine (nonivamide).

| Test substance | T/C [%] | Standard deviation |
|---|---|---|
| Control | 100.00 | 22.31 |
| EtOH control | 100.00 | 10.80 |
| 50 mM KCl | 153.48 | 9.9 |
| N-Nonanoylvanillylamine 0.01 μM | 191.35 | 98.18 |
| N-Nonanoylvanillylamine 0.1 μM | 226.44 | 39.71 |
| N-Nonanoylvanillylamine 1 μM | 272.12 | 115.53 |
| N-Nonanoylvanillylamine 10 μM | 185.65 | 72.76 |

At a concentration of N-nonanoylvanillylamine of 0.1 μM and above, the amount of serotonin released by SH-SY5Y cells increases highly significantly, i.e. p<0.005.

### Example 4

### Release of Serotonin in an Experimental Cell System with N-nonanoylvanillylamine and a TRPV1 Inhibitor

After stimulation of 1.25*10⁶ human neuroblastoma cells (SH-SY5Y) for 3 minutes with 300 μl of Krebs-Ringer HEPES buffer (0.1% ascorbic acid, pH 6.2), with or without addition of N-nonanoylvanillylamine (so that a concentration of 1 μM N-nonanoylvanillylamine is established in the Krebs-Ringer HEPES buffer) in combination with different application-relevant amounts of the selective TRPV1 inhibitor trans-4-tert-butylcyclohexanol, the serotonin content is determined by means of an enzyme-based detection method (serotonin ELISA sensitive, DLD Diagnostica, Hamburg, Germany). The cells are lysed with a buffer containing sodium lauryl sarcosinate, and the DNA content is determined by means of a NanoQuant plate in a plate reader (Tecan, Ménnendorf, Switzerland) for normalisation of the serotonin release.

TABLE 4
Data relating to the release of serotonin by SH-SY5Y cells following
stimulation with 1 μM N-nonanoylvanillylamine or different
concentrations of N-nonanoylvanillylamine (nonivamide).

| Test substance | T/C [%] | Standard deviation |
|---|---|---|
| Control | 100.00 | 8.97 |
| N-Nonanoylvanillylamine 1 μM | 151.75 | 35.46 |
| N-Nonanoylvanillylamine 1 μM + 25 μM | 154.86 | 28.13 |
| trans-4-tert-butylcyclohexanol | | |
| N-Nonanoylvanillylamine 1μM + 50 μM | 128.74 | 13.12 |
| trans-4-tert-butylcyclohexanol | | |
| N-Nonanoylvanillylamine 1 μM + 80 μM | 140.81 | 24.92 |
| trans-4-tert-butylcyclohexanol | | |
| N-Nonanoylvanillylamine 1 μM + 200 μM | 181.93 | 49.64 |
| trans-4-tert-butylcyclohexanol | | |
| 25 μM trans-4-tert-butylcyclohexanol | 91.07 | 15.20 |
| 50 μM trans-4-tert-butylcyclohexanol | 101.39 | 16.59 |
| 80 μM trans-4-tert-butylcyclohexanol | 84.129 | 10.73 |
| 200 μM trans-4-tert-butylcyclohexanol | 110.99 | 13.76 |

The present data show that the release of serotonin in SH-SY5Y cells by N-nonanoylvanillylamine is not significantly influenced by addition of the selective TRPV1 inhibitor trans-4-tert-butylcyclohexanol. The TRPV1 receptor, which is responsible for the pungent taste, is accordingly not involved in the release of serotonin caused by N-nonanoylvanillylamine in SH-SY5Y cells.

### APPLICATION EXAMPLES

### Application Example 1

### Refreshing Drink (Containing Sugar, Reduced-Ccalorie, Calorie-Free)

| Ingredient | Amount used in wt. % | | | | | | |
|---|---|---|---|---|---|---|---|
| Preparation | A | B | C | D | E | F | G |
| Sugar (sucrose) | 10 | 10 | 7 | — | — | 8 | 7 |
| Glucose/fructose | — | — | — | — | 10 | — | — |
| syrup from | | | | | | | |
| maize, | | | | | | | |
| containing | | | | | | | |
| 55 wt. % fructose | | | | | | | |
| Rebaudioside A | — | — | 0.02 | 0.05 | — | — | — |
| 95% | | | | | | | |
| Citric acid | 0.15 | 0.15 | 0.06 | 0.15 | 0.15 | 0.15 | 0.15 |
| Phosphoric acid | — | — | 0.07 | — | — | — | — |
| Caramel colour | — | — | 0.14 | — | — | — | — |
| Caffeine | — | — | 0.01 | — | — | — | — |
| Lemon flavour | 0.1 | 0.05 | — | 0.1 | 0.1 | 0.1 | 0.1 |
| Limonene | — | 0.05 | — | — | — | — | — |
| flavour | | | | | | | |
| “Cola”-type drink | — | — | 0.05 | — | — | — | — |
| emulsion | | | | | | | |
| Phloretin | — | — | 0.002 | 0.003 | — | 0.002 | 0.001 |
| Hesperetin | — | — | 0.001 | 0.002 | — | — | 0.002 |
| Extract from | — | — | — | — | — | 0.01 | — |
| Rubus | | | | | | | |
| suavissimus, | | | | | | | |
| containing 5 | | | | | | | |
| wt. % | | | | | | | |
| rubusoside, | | | | | | | |
| based on the | | | | | | | |
| total weight of | | | | | | | |
| the extract | | | | | | | |
| Homoeriodictyol | — | — | 0.005 | 0.005 | — | — | — |
| sodium salt | | | | | | | |
| N-Nonanoyl- | 0.000003 | 0.0001 | 0.00005 | 0.00001 | 0.000003 | 0.0001 | 0.00005 |
| vanillylamine | | | | | | | |
| Eriodictyol | — | 0.0100 | 0.0100 | — | — | 0.0100 | 0.0100 |

| | |
|---|---|
| Water | make up to 100 |

The ingredients were mixed in the indicated order and made up to 100% with water. The mixtures are introduced into glass bottles and carbonated.

### Application Example 2

### Use in a Chewing Gum

| | | Amount used in wt. % | |
|---|---|---|---|
| Part | Ingredient | A | B |
| A | Chewing-gum base, “Jagum T” | 30.4899 | 30.49999 |
| B | Sorbitol, powdered | 39.00 | 39.00 |
| | Isomalt ® (Palatinit GmbH) | 9.50 | 9.50 |
| | Xylitol | 2.00 | 2.00 |
| | Mannitol | 3.00 | 3.00 |
| | Aspartame ® | 0.10 | 0.10 |
| | Acesulfam ® K | 0.10 | 0.10 |
| | Emulgum ® (Colloides Naturels, Inc.) | 0.30 | 0.30 |
| C | Sorbitol, 70% | 14.00 | 14.00 |
| | Glycerol | 1.00 | 1.00 |
| D | Peppermint flavour | 0.5 | 0.5 |
| | N-Nonanoylvanillylamine | 0.0001 | 0.00001 |
| | Eriodictyol | 0.0100 | — |

Parts A to D are mixed and kneaded intensively. The crude mass can be processed, for example, in the form of thin strips into ready-to-eat chewing gum.

### Application Example 3

### Use in Hard Caramels

| | Content (wt. %) | | | |
|---|---|---|---|---|
| Ingredient | A | B | C | D |
| Sugar | 75.40 | — | — | — |
| Palatinit, type M | — | 74.00 | 75.50 | 75.00 |
| Citric acid | 0.5 | 1.0 | 0.5 | — |
| Colouring yellow | — | 0.01 | — | — |
| Colouring red | — | — | 0.01 | — |
| Colouring blue | 0.01 | — | — | 0.01 |
| Peppermint flavour | 0.1 | — | — | 0.1 |
| Lemon flavour | — | 0.1 | — | — |
| Red fruit flavour | — | — | 0.1 | — |
| Rebaudioside A 98% | — | 0.040 | — | 0.040 |
| Balansin A according to [SY317] | — | 0.005 | 0.010 | 0.005 |
| Hesperetin | — | 0.001 | — | 0.001 |
| Phloretin | — | 0.002 | — | — |
| N-Nonanoylvanillylamine | 0.0001 | 0.00001 | 0.000005 | 0.0001 |
| Eriodictyol | 0.0100 | — | — | — |
| para-4-tert-Butylcyclohexanol | — | — | — | 0.0100 |
| Water | ad 100 | ad 100 | ad 100 | ad 100 |

Palatinit, or the sugar, was mixed with water, where appropriate after addition of the citric acid, and the mixture was melted at 165° C. and then cooled to 115° C. The flavour and the other constituents were added and, after thorough mixing, the mixture was poured into moulds, removed from the moulds after solidifying and then packaged individually.

### Application Example 4

### Low-Fat Yoghurts

| | Preparation (amounts in wt. %) | | | |
|---|---|---|---|---|
| Ingredient | A | B | C | D |
| Sucrose | 10 | 8 | 6 | — |
| Rebaudioside A 98% | — | — | — | 0.050 |
| Extract from Rubus | — | 0.010 | 0.010 | — |
| suavissimus, containing | | | | |
| 5 wt. % rubusoside, based | | | | |
| on the total weight of the | | | | |
| extract, for example of | | | | |
| plant extract | | | | |
| Hesperetin | — | 0.001 | 0.001 | 0.002 |
| Phloretin | — | — | 0.002 | 0.002 |
| Homoeriodictyol sodium | — | — | — | 0.005 |
| salt | | | | |
| N-Nonanoylvanillylamine | 0.0001 | 0.0001 | 0.000005 | 0.0001 |
| Eriodictyol | | 0.0100 | | 0.0100 |

| | |
|---|---|
| Yoghurt, 0.1% fat | make up to 100% |

The ingredients were mixed and cooled to 5° C.

### Application Example 5

### Fruit Gums

| | Preparation (amounts in wt. %) | |
|---|---|---|
| Ingredient | A | B |
| Sucrose | 34.50 | 8.20 |
| Glucose syrup, DE 40 | 31.89 | 30.09 |
| Iso Syrup C* Tru Sweet 01750 | 1.50 | 2.10 |
| (Cerestar GmbH) | | |
| Gelatine 240 Bloom | 8.20 | 9.40 |
| Polydextrose (Litesse ® Ultra, | — | 24.40 |
| Danisco Cultor GmbH) | | |
| Yellow and red colouring | 0.01 | 0.01 |
| Citric acid | 0.20 | |
| Cherry flavour, containing 1 wt. % | — | 0.10 |
| hesperetin 2 and 0.3 wt. % phloretin, | | |
| based on the flavour | | |
| N-Nonanoylvanillylamine | 0.0001 | 0.00001 |
| Eriodictyol | 0.0100 | |
| Water | ad 100 | ad 100 |

Polydextrose is a polysaccharide of low calorific value which does not have a sweet taste.

1. N-Nonanoylvanillylamine for use in a therapeutic or non-therapeutic method
(a) as an agent for reducing the appetite,
preferably for reducing the caloric intake and hence preferably for therapeutic or non-therapeutic weight reduction,
and/or
(b) as an agent for imparting a feeling of fullness,
preferably for reducing the caloric intake and hence preferably for therapeutic or non-therapeutic weight reduction,
and/or
(c) as a mood enhancer.

2. (canceled)

3. N-Nonanoylvanillylamine for use according to claim 1, wherein the N-nonanoylvanillylamine is used as a constituent of an orally consumable product, wherein
the N-nonanoylvanillylamine is present in a concentration of 1 mg/kg or less, based on the total mass of the orally consumable product, preferably in a concentration of 0.50 mg/kg or less, particularly preferably in a concentration of 0.10 mg/kg or less, most particularly preferably in a concentration of 0.05 mg/kg or less,
and/or
the orally consumable product comprises no capsaicin, preferably no capsaicin and, apart from N-nonanoylvanillylamine, no further capsaicinoids.

4. Method
(a) for the therapeutic or non-therapeutic reduction of the appetite, preferably for the non-therapeutic reduction of the caloric intake and hence preferably for therapeutic or non-therapeutic weight reduction,
and/or
(b) for the therapeutic or non-therapeutic imparting of a feeling of fullness, preferably for the therapeutic or non-therapeutic reduction of the caloric intake and hence preferably for non-therapeutic weight reduction,
and/or
(c) for enhancing the mood, comprising the following step:
administration of N-nonanoylvanillylamine to a human or animal subject in an amount that (i) reduces the appetite and/or (ii) brings about a feeling of fullness and/or (iii) enhances the mood.

5. Substance mixture comprising N-nonanoylvanillylamine and one or more further substances, for use in the a therapeutic or non-therapeutic method according to claim 1, wherein the N-nonanoylvanillylamine contained in the substance mixture is used in such a manner that it reduces the appetite and/or causes a feeling of fullness and/or enhances the mood.

6. (canceled)

7. Substance mixture for use according to claim 5,
wherein the substance mixture is an orally consumable product or a constituent of an orally consumable product,
the orally consumable product comprises the N-nonanoylvanillylamine preferably in a concentration of less than 1 mg/kg of orally consumable product, based on the total mass of the foodstuff, feed or medicament,
preferably in a concentration of 0.50 mg/kg or less, particularly preferably in a concentration of 0.10 mg/kg or less, most particularly preferably in a concentration of 0.05 mg/kg or less,
the substance mixture preferably
comprises a maximum amount of capsaicin such that the ratio by mass capsaicin: N-nonanoylvanillylamine is 100:1, preferably 50:1, more preferably 20:1, more preferably 1:1, more preferably 1:10 and more preferably 1:100,
the substance mixture preferably comprises no capsaicin, more preferably no capsaicin and, apart from N-nonanoylvanillylamine, no further capsaicinoids.

8. Orally consumable product comprising N-nonanoylvanillylamine and one or more further substances, wherein the N-nonanoylvanillylamine is present
in a concentration that reduces the appetite and/or brings about a feeling of fullness
and/or
in a concentration that enhances the mood
and at the same time
in a concentration of 1 mg/kg or less, based on the total mass of the orally consumable product, preferably in a concentration of 0.50 mg/kg of orally consumable product or less, particularly preferably in a concentration of 0.10 mg/kg or less, most particularly preferably in a concentration of 0.05 mg/kg or less,
and at the same time
in a concentration of at least 0.001 mg/kg or more, based on the total mass of the orally consumable product, preferably in a concentration of 0.005 mg/kg of orally consumable product or more, most particularly preferably in a concentration of 0.01 mg/kg or more, and
the orally consumable product comprises no capsaicin,
preferably no capsaicin and, apart from N-nonanoylvanillylamine, no further capsaicinoids,
wherein the orally consumable product contains not more than 200 kcal/100 g of orally consumable product, preferably not more than 100 kcal/100 g, particularly preferably not more than 40 kcal/100 g.

9. Orally consumable product according to claim 8, wherein the orally consumable product is selected from the group comprising confectionery, non-alcoholic drinks, instant drinks, cereal products, dairy products, products made from soy protein or other soybean fractions, sweetener preparations, sweetener tablets and sweetener sachets, ice-cream, dragées,
and/or
wherein the orally consumable product comprises (a) one, two or more sweeteners and/or (b) one, two or more thickeners.

10. Orally consumable product according to claim 8, wherein the orally consumable product comprises milk thickened with lactic acid bacteria and/or cream thickened with lactic acid bacteria and preferably is selected from the group comprising orally consumable products having a fat content of 4.0 wt. % or less, preferably of 1.5 wt. % or less, particularly preferably 0.5 wt. % or less, in each case based on the total weight of the orally consumable product,
and/or
is selected from the group comprising yoghurt, kefir and quark,
and/or
wherein the orally consumable product contains not more than 150 kcal/100 g, preferably not more than 100 kcal/100 g, particularly preferably not more than 75 kcal/100 g, particularly preferably not more than 50 kcal/100 g.

11. Orally consumable product according to claim 10, wherein the orally consumable product comprises fruits and/or fruit preparations and/or
the orally consumable product comprises (i) sugars and/or (ii) thickeners and/or (iii) gelling agents and/or (iv) sweeteners and/or (v) flavours and/or (vi) preservatives.

12. Orally consumable product according to claim 9, wherein the orally consumable product comprises a probiotic, wherein the probiotic is preferably selected from the group comprising Bifidobacterium animalis subsp. lactis BB-12, Bifidobacterium animalis subsp. lactis DN-173 010, Bifidobacterium animalis subsp. lactis HNO19, Lactobacillus acidophilus LA5, Lactobacillus acidophilus NCFM, Lactobacillus johnsonii Lal, Lactobacillus casei immunitass/defensis, Lactobacillus casei Shirota (DSM 20312), Lactobacillus casei CRL431, Lactobacillus reuteri (ATCC 55730) and Lactobacillus rhamnosus (ATCC 53013).

13. Orally consumable product according to claim 8, wherein the orally consumable product comprises a chewing-gum base, wherein the chewing- gum base is preferably selected from the group comprising natural resins, the natural latex chicle, polyvinyl acetate (PVA), polyethylene, low molecular weight polyisobutene (Pm), medium molecular weight polyisobutene (PIB), polybutadiene, isobutene-isoprene copolymers (butyl rubber), polyvinyethyl ether (PVE), polyvinylbutyl ether, copolymers of vinyl esters and vinyl ethers, styrene-butadiene copolymers (styrene-butadiene rubber, SBR), vinyl elastomers, vinyl elastomers based on vinyl acetate/vinyl laurate, vinyl elastomers based on vinyl acetate/vinyl stearate and vinyl elastomers based on ethylene/vinyl acetate.

14. Orally consumable product according to claim 8, wherein the orally consumable product is a drink,
the drink preferably has a sugar content of 30 g/100 ml of the orally consumable product or less, preferably of 15 mg/100 ml or less, particularly preferably 5 g/100 ml or less, particularly preferably comprises no sugar,
and/or
the drink contains no ethanol or not more than 0.1 percent by volume ethanol, based on the volume of the drink,
the drink is preferably a carbonated drink or an uncarbonated drink.

15. Substance mixture according to claim 5, additionally comprising one or more TRPV1 inhibitors, preferably selected from the group comprising trans-tert-butylcyclohexanol and eriodictyol.

16. Orally consumable product according to claim 9, wherein the orally consumable product comprises milk thickened with lactic acid bacteria and/or cream thickened with lactic acid bacteria and preferably
is selected from the group comprising orally consumable products having a fat content of 4.0 wt. % or less, preferably of 1.5 wt. % or less, particularly preferably 0.5 wt. % or less, in each case based on the total weight of the orally consumable product,
and/or
is selected from the group comprising yoghurt, kefir and quark,
and/or
wherein the orally consumable product contains not more than 150 kcal/100 g, preferably not more than 100 kcal/100 g, particularly preferably not more than 75 kcal/100 g, particularly preferably not more than 50 kcal/100 g.

17. Orally consumable product according to claim 16, wherein the orally consumable product comprises a probiotic, wherein the probiotic is preferably selected from the group comprising Bifidobacterium animalis subsp. lactis BB-12, Bifidobacterium animalis subsp. lactis DN-173 010, Bifidobacterium animalis subsp. lactis HNO19, Lactobacillus acidophilus LAS, Lactobacillus acidophilus NCFM, Lactobacillus johnsonii Lal, Lactobacillus casei immunitass/defensis, Lactobacillus casei Shirota (DSM 20312), Lactobacillus casei CRL431, Lactobacillus reuteri (ATCC 55730) and Lactobacillus rhamnosus (ATCC 53013).

18. Orally consumable product according to claim 11, wherein the orally consumable product comprises a probiotic, wherein the probiotic is preferably selected from the group comprising Bifidobacterium animalis subsp. lactis BB-12, Bifidobacterium animalis subsp. lactis DN-173 010, Bifidobacterium animalis subsp. lactis HNO19, Lactobacillus acidophilus LA5, Lactobacillus acidophilus NCFM, Lactobacillus johnsonii Lal, Lactobacillus casei immunitass/defensis, Lactobacillus casei Shirota (DSM 20312), Lactobacillus casei CRL431, Lactobacillus reuteri (ATCC 55730) and Lactobacillus rhamnosus (ATCC 53013).

19. Orally consumable product according to claim 10, wherein the orally consumable product comprises a probiotic, wherein the probiotic is preferably selected from the group comprising Bifidobacterium animalis subsp. lactis BB-12, Bifidobacterium animalis subsp. lactis DN-173 010, Bifidobacterium animalis subsp. lactis HNO19, Lactobacillus acidophilus LA5, Lactobacillus acidophilus NCFM, Lactobacillus johnsonii Lal, Lactobacillus casei immunitass/defensis, Lactobacillus casei Shirota (DSM 20312), Lactobacillus casei CRL431, Lactobacillus reuteri (ATCC 55730) and Lactobacillus rhamnosus (ATCC 53013).

20. Orally consumable product according to claim 9, wherein the orally consumable product comprises a chewing-gum base, wherein the chewing- gum base is preferably selected from the group comprising natural resins, the natural latex chicle, polyvinyl acetate (PVA), polyethylene, low molecular weight polyisobutene (PM), medium molecular weight polyisobutene (PIB), polybutadiene, isobutene-isoprene copolymers (butyl rubber), polyvinyethyl ether (PVE), polyvinylbutyl ether, copolymers of vinyl esters and vinyl ethers, styrene-butadiene copolymers (styrene-butadiene rubber, SBR), vinyl elastomers, vinyl elastomers based on vinyl acetate/vinyl laurate, vinyl elastomers based on vinyl acetate/vinyl stearate and vinyl elastomers based on ethylene/vinyl acetate.

21. Orally consumable product according to claim 9, wherein the orally consumable product is a drink,
the drink preferably has a sugar content of 30 g/100 ml of the orally consumable product or less, preferably of 15 mg/100 ml or less, particularly preferably 5 g/100 ml or less, particularly preferably comprises no sugar,
and/or
the drink contains no ethanol or not more than 0.1 percent by volume ethanol, based on the volume of the drink,
the drink is preferably a carbonated drink or an uncarbonated drink.

22. Orally consumable product according to claim 8, additionally comprising one or more TRPV1 inhibitors, preferably selected from the group comprising trans-tert-butylcyclohexanol and eriodictyol.
